(19) United States
(12) Patent Application Publication
(10) Pub. No.: US 2023/0190007 A1
(43) Pub. Date: Jun. 22, 2023

(54) AUTOMATIC PIVOTING BASSINET FOR THE PREVENTION AND TREATMENT OF PLAGIOCEPHALY

(71) Applicant: Karim Romman, Pasadena, TX (US)

(72) Inventor: Karim Romman, Pasadena, TX (US)

(21) Appl. No.: 17/937,881

(22) Filed: Oct. 4, 2022

Related U.S. Application Data
(60) Provisional application No. 63/291,542, filed on Dec. 20, 2021.

(51) Int. Cl. A47D 9/02

(52) U.S. Cl. CPC A47D 9/057

(57) ABSTRACT

A bassinet configured to prevent and treat plagiocephaly of an infant via an automated pivoting floor platform (bed) via an actuating motor disposed of in communication with a controlling timer, a power source, and at least one support. The motor is preferably insulated to ensure a quiet operation, minimizing the possibility of waking the infant during use. A soothing sound machine is preferably integrated into the apparatus and may be optionally activated at the user's will for a more restful sleep. The floor platform is preferably configured to tilt 15-20 degrees to either side, slowly, in an oscillating manner in intervals of 15 minutes. Straps present in communication with the floor platform and/or side walls of the bassinet facilitate the connection of a swaddle or onesie to the floor platform, ensuring the infant remains in position even when tilted. Tilting of the head is allowed, preventing flatting of the head in a single area due to the automatic movement of the head during the sleep session.

### CONTINUITY

This application is a non-provisional patent application of provisional patent application No. 63/291,542, filed on Dec. 20, 2021, and priority is claimed thereto.

### FIELD OF THE PRESENT INVENTION

The present invention relates to the field of infant safety devices and, more specifically, relates to an automatic pivoting bassinet configured to gently shift the placement of an infant's head during sleep to facilitate the prevention and/or treatment of plagiocephaly.

### BACKGROUND OF THE PRESENT INVENTION

“SIDS” or “SUDDEN INFANT DEATH SYNDROME” is the unexplained death during the sleep of healthy babies younger than two to six months of age. SIDS causes about 3400 babies to die suddenly every year in the US. The cause of death is unknown, but researchers found that the most important factor in preventing SIDS is to put the baby on their back to sleep

Following that, the American Academy of Pediatrics changed its recommendation in 1992 and announced that babies must be placed on their backs to sleep. AAP upheld the recommendation every year since then. This was followed by national public education campaign to educate and raise awareness about SIDS risk reduction. Campaigns by AAP, the SIDS alliance, maternal and child health bureau of the health resources and services administration, and others raised public awareness. That worked very well, and reduced SIDS significantly. However, the prominent practice of having babies sleep on their backs has resulted in the unfortunate occurrence of cases of plagiocephaly.

Positional plagiocephaly is a condition in which specific areas of an infant's head develop an abnormally flattened shape and appearance. Occipital plagiocephaly causes a flattening of one side of the back of the head and is often a result of the infant consistently lying on his or her back. A flat area may develop very quickly over several months. Physically, the infant may have one ear that is shifted forward. In more severe cases, the infant may have forehead or cheek protrusion on the flat side of his or her head. Craniosynostosis is another form of plagiocephaly. It is a deformity caused by the premature closure of the fibrous joints between the bones of the infant skull and often requires surgical correction. The condition of plagiocephaly affects 20-25% of babies within the first few months of life. Additionally, the condition is not only cosmetic but 19% of these babies have developmental delays.

The public has been heeding the advice and responding to the campaigns by putting the babies on their backs. This succeeded in saving a lot of infants' lives in the last two decades. This, however, resulted in many infants developing plagiocephaly because they spend all their sleeping time on their backs. Because these babies have soft bones, this caused parts of the head to become flat at the pressure points, and that affected the symmetry of the face.

The recommendation to treat plagiocephaly so far has included providing the infant with plenty of supervised playtimes on his or her tummy to strengthen neck, shoulder, and arm muscles, changing the direction the baby is lying in the crib regularly to ensure he or she is not always resting on the same part of the head, and avoiding too much time in car seats, carriers and bouncers while the baby is awake. Additionally, parents are encouraged to frequently get “cuddle time” during the day by holding the baby upright over one shoulder. When holding, feeding, or carrying an infant, make sure that there is no undue pressure placed on the flat side of the head. Changing the infant's head position from side to side during feeding time can also be helpful. Likewise, changing the location of the baby's crib in the room so that he or she has to look in different directions to see the door or the window may also help.

Unfortunately, some of this advice is easier to heed than others. Therefore, these treatments have had limited success, and many of these babies with symptoms of plagiocephaly are referred to neurosurgeons. This is an appropriate referral for craniosynostosis type of plagiocephaly. Infants diagnosed with positional plagiocephaly also go through the referral and end up needing skull X-Rays, CT scans, and ultimately referrals to a plastic surgeon or neurosurgeon. They also get a helmet to correct the shape of the head. Helmets and other methods do not always completely correct the deformity. If there were an easier means to treat plagiocephaly over time at home before neurosurgery is required, the infant's health would be aided, and the parents' stress could be minimized.

Patent EP1343400B1 by John H. et al. dated May 19, 2010, which is an Infant Rocking Apparatus, an infant support device with a rocking mechanism, has lifters whose upper and lower ends are connected to the side and base of infant support, respectively, for moving support along two sides. An infant rocking apparatus (2) has infant support (3) with trough (6) extending centrally between the side portions (8,10) and a pair of lifters (13). The upper and lower ends of the lifters are connected to side portions (8,10) and the base of the support, respectively, for moving the support along two sides. Richard's design may be enough to support an infant but has no further function. By the stated features, it does generally support an infant alongside their movements. Still, John H.'s design is a general feature that focuses on an infant's body movement, dividing the support.

Patent US20120036635A1 by Robin et al., published on Feb. 16, 2012, by Robin et al. depicts a baby carrier rocking system with different rocking speed settings that will rock any bottom-shaped carrier profile from flat to curved. An electric motor drives an offset link that raises and lowers four supporting arms underneath the baby carrier, thus rocking the carrier back and forth from its center pivoting position. An improved portable baby carrier rocking system which allows almost all existing car seat carriers, baby carriers, and baby bassinets to be attached to it. This baby carrier rocker mounts underneath the baby carrier and attaches to it by a simple strap. This baby carrier rocker can be battery operated or powered by an external power supply. It has different rocking speed settings and will rock any bottom-shaped carrier profile from flat to curved. An electric motor drives an offset link in which raises and lowers four supporting arms underneath the baby carrier, thus rocking the carrier back and forth from its center pivoting position. This baby carrier rocker has extendable and retractable legs which flip out for greater rocker stability. Its novel compact size makes this rocker easily transportable and can be used in small, confined areas like restaurants, camping tents, crowded rooms, etc. Robin's design depicts it as a sturdy and hassle-free solution as it can rock any carrier of a particular shape, but it has no regularity when it comes to soothing and movement control for an infant, which means that no support is particularly present.

Michael et al.'s U.S. Pat. No. 8,950,020B2, published Feb. 10, 2015, features an automatic bassinet rocking device that imparts a rocking motion to a bassinet or rocking crib. A housing is supported above the rocker base of the bassinet on adjustable legs so that the device straddles the curved rocking base. A reciprocating rod descends from the housing and attaches to a strap or clamp at an anchor point on the rocker base. An oscillatory drive inside the housing imparts a reciprocating motion via the reciprocating rod onto the rocker base. This device is designed and does provide rocking motions while in use for an infant. These rocking motions are supposed to help the infant by providing an optimal feeling of rocking them to sleep. Still, Michael's design does not offer any resolution regarding movement and head and body support.

Richard et al.'s Patent US20210100368A1 was published on Sep. 18, 2020 An infant care apparatus includes a base. A drive mechanism and a vibratory mechanism are coupled to the base. A movable stage is movably mounted to the base. Infant support is coupled to the movable stage. The drive mechanism imparts a first cyclic motion to the movable stage and a second cyclic motion to at least part of the movable stage independent of the first cyclic motion and to the vibratory mechanism, the vibration motor vibrates the movable stage. The infant support is coupled to the movable stage and moves cyclically in both the first and second cyclic motions. The controller is configured to move the infant support in a selectively variable motion profile with selectable vibration modes selected from different selectively variable motion profiles and selectively different vibration modes for each of the different selectable variable motion profiles. The apparatus is designed as a sophisticated apparatus that provides comfort and support for an infant by having features that enable it to play white noises for the infant or secure the infant's movement and position. Still, besides all this, Richard's design does not offer any support to the infant's movement.

### SUMMARY OF THE PRESENT INVENTION

The present invention is an automatic pivoting bassinet configured to prevent plagiocephaly and treat existing mild cases of plagiocephaly. The apparatus is configured to pivot 15 to 20 degrees clockwise and 15 to 20 degrees counterclockwise (i.e., to the right and to the left) via an electric motor. The motor is preferably insulated to ensure a quiet operation to prevent disturbance of the infant during sleep. Automation ensures that the floor (bed) of the bassinet pivots once every fifteen minutes; however, the frequency of the motion may be adjusted by the end user if desired.

It is an object of the present invention to provide a better alternative to placing the baby in “Cranial Remodeling Helmets” after 3D scanning starting from four months to 18 months. Helmets cost several thousand and may be needed for a year or 14 months.

Further, it is an object of the present invention to remove the need for 95% of the helmets prescribed by dealing with the cause itself, as well as to provide treatment to easily correct mild cases of plagiocephaly at home. The cause is keeping the babies with soft bones on the back for long periods resulting in the force of gravity flattening the head at the pressure points, ultimately resulting in the deformity. In response, the present invention employs the force of gravity to correct the deformity, removing the need for surgery or corrective helmets.

The following brief and detailed descriptions of the drawings are provided to explain possible embodiments of the present invention but are not provided to limit the scope of the present invention as expressed herein in this summary section.

### BRIEF DESCRIPTION OF THE DRAWINGS

The accompanying drawings, which are incorporated herein and form a part of the specification, illustrate the present invention and, together with the description, further explain the invention's principles and enable a person skilled in the pertinent art to make and use the invention.

The present invention will be better understood with reference to the appended drawing sheets, wherein:

FIG. 1 depicts a view of the bed of the bassinet of the present invention, showing an infant secured (swaddled) within the bassinet for sleep.

FIG. 2 shows a diagram detailing the pressure distribution, as enacted by gravity, on the infant's skull during sleep as the bassinet of the present invention is pivoted throughout the night.

FIG. 3 exhibits a view of a first embodiment of the present invention as depicted from the side.

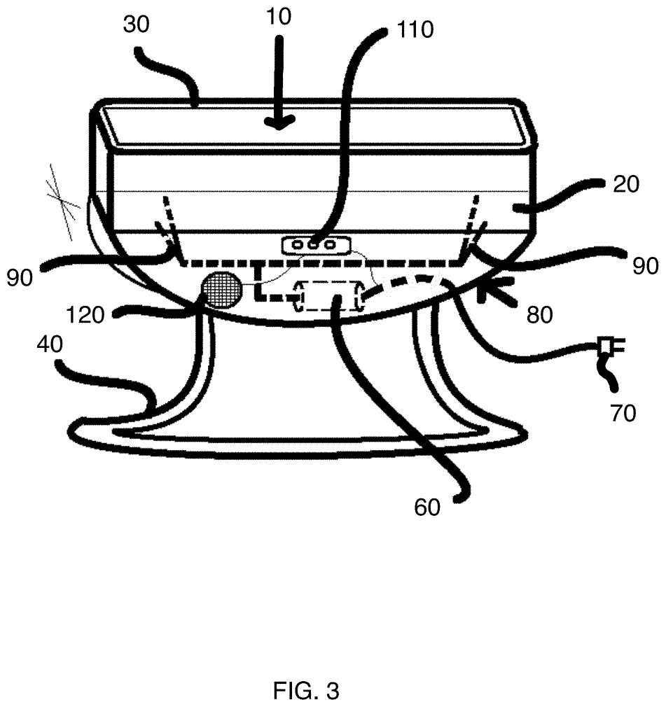

FIG. 4 details a flowchart describing the process of use of the present invention by a parent for their infant.

FIG. 5 depicts a view of the straps and buckles of the present invention as seen from above.

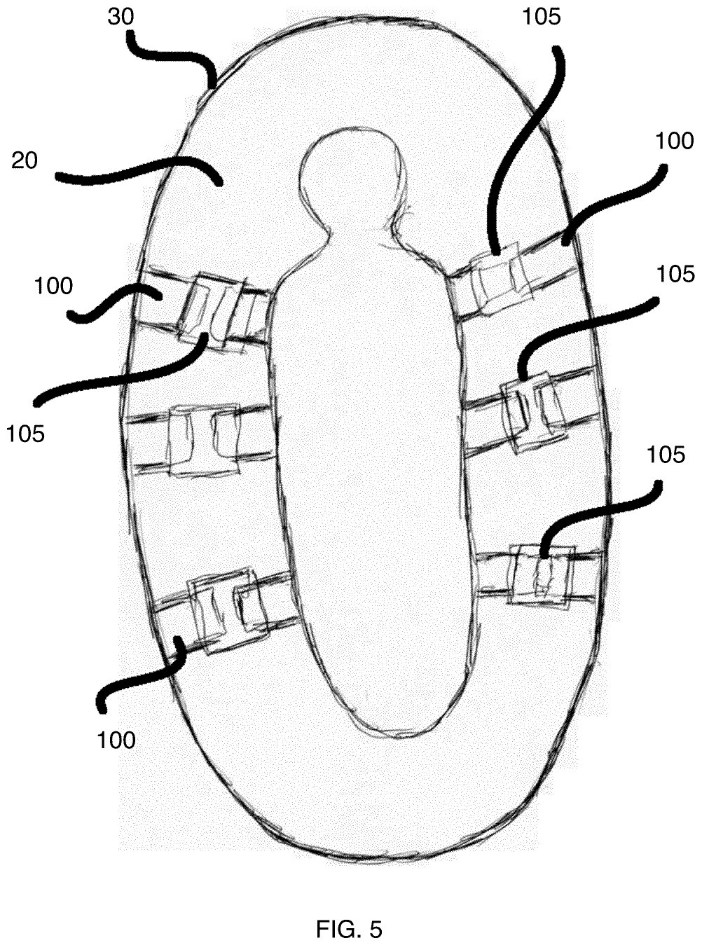

### DETAILED DESCRIPTION OF THE PREFERRED EMBODIMENT

The present specification discloses one or more embodiments that incorporate the features of the invention. The disclosed embodiment(s) merely exemplify the invention. The scope of the invention is not limited to the disclosed embodiment(s).

References in the specification to “one embodiment,” “an embodiment,” “an example embodiment,” etc., indicate that the embodiment described may include a particular feature, structure, or characteristic, but every embodiment may not necessarily include the particular feature, structure or characteristic. Moreover, such phrases are not necessarily referring to the same embodiment; Further, when a particular feature, structure, or characteristic is described in connection with an embodiment, it is submitted that it is within the knowledge of one skilled in the art to effect such feature, structure, or characteristic in connection with other embodiments whether or not explicitly described.

The present invention is an automatic pivoting bassinet configured to both prevent and treat plagiocephaly. The present invention is a bassinet equipped with a bed area (10) having a floor platform (20), surrounding wall (30), and supports (40). The floor platform (20) traditionally functions as a mattress or bed for an infant. The supports (40) are preferably disposed of in communication with a bottom (50) of the floor platform (20), which provides even and adequate support for the weight of the floor platform (20) and infant during use.

An electric motor (60) disposed of in communication with a power source (70) is present within a housing (80) of the present invention. The power source (70) is preferably an AC power outlet, however, some embodiments may be equipped with batteries. The electric motor (60) is preferably affixed to at least one actuating aim (90), which is disposed of in communication with the floor platform (20) to facilitate actuation (tilting) of the floor platform (20). The floor platform (20) is configured to be angled automatically via the electric motor (60) at specific intervals (preferably every 15 minutes). Angles at which the floor platform (20) may be disposed include −15 degrees, 0 degrees, and 15 degrees, amounting to a slowly oscillating clockwise/counterclockwise motion.

In some embodiments of the present invention, the floor platform (20) may pivot up to 20 degrees in either direction. Movement of the floor platform (20) allows the distribution of the force of gravity to be applied evenly to the head of the infant throughout the night, preventing and/or treating the flattening of the head. The electric motor (60) is configured to operate very slowly and smoothly, ensuring a gentle rotation of the head of the infant. The frequency of the actuation of the electric motor (60) is preferably once every fifteen minutes, however, in preferred embodiments of the present invention, the parent or guardian of the infant may alter the frequency if desired. It should be noted that the degree and frequency of the actuation of the electric motor (60) are dependent on the shape of the infant's head and the severity of the case of plagiocephaly.

In some alternate embodiments of the present invention, a mechanical motor is employed in place of an electric motor (60). In other alternate embodiments, the floor platform (20) is pivoted manually by the parent or guardian via a hand crank or lever. Further, in some alternate embodiments of the present invention, the entirety of the bassinet pivots about a primary axis to facilitate movement of the head of the infant during sleep.

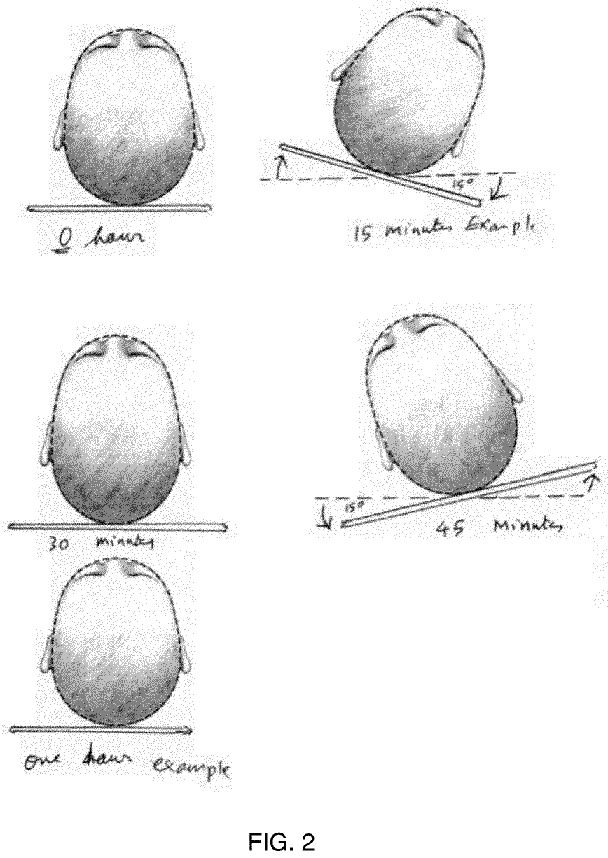

As shown in FIG. 2, pressure distribution, as enacted by the gravitational force, is applied evenly when the present invention is properly used consistently over time, preventing the exertion of sustained pressure in one position which prevents flattening of that area of the head.

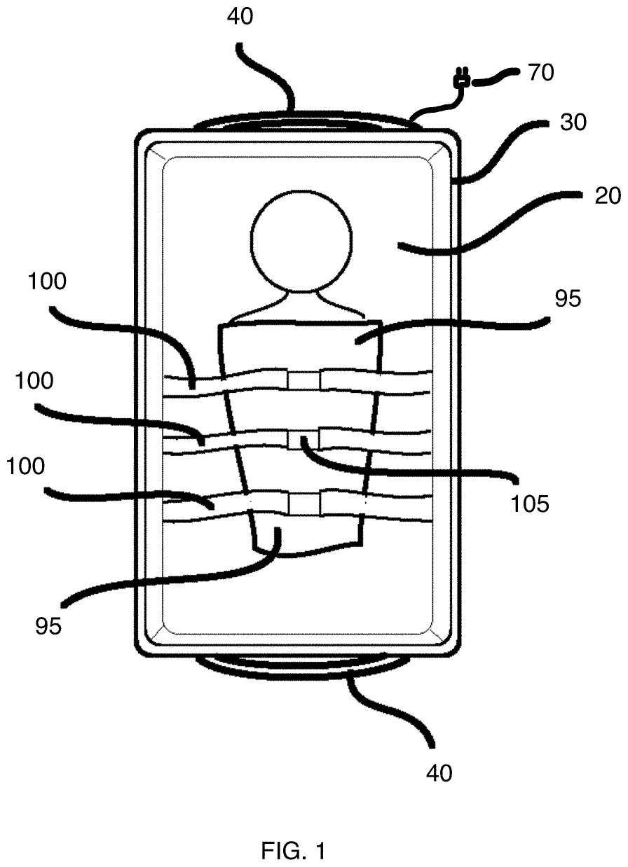

Retaining straps (100) are preferably present, either in communication with a specially designed onesie or in communication with the floor platform (20) itself. The retaining straps (100), as shown in FIG. 1, ensure that the infant remains in place, even during the pivoting process. Clasps or buckles (105) may be present on the onesie WE NEED TO DETAIL AND SHOW THE ONESIE IF ESPECIALLY RELEVANT or on a special swaddle (95) WE HAVE TO EXPLAIN 95 which affix to the straps (100) connected to the floor platform (20). The onesies and/or swaddles are preferably available in multiple sizes to accommodate differing sizes of babies. The retaining straps (100) are preferably similar to those of car seat straps. Both options allow for easy access to the baby and prevents overheating.

Additionally, some embodiments of the present invention are preferably equipped with a soothing sound feature which is controlled via buttons (110) found on the front or side of the bassinet. At least one speaker (120) facilitates the playback of soothing sounds to help the infant to sleep best. The sounds may be toggled on or off via a power button, and multiple sound options are preferably available, including but not limited to white noise, heartbeat noise, wave noise, and other similar noises.

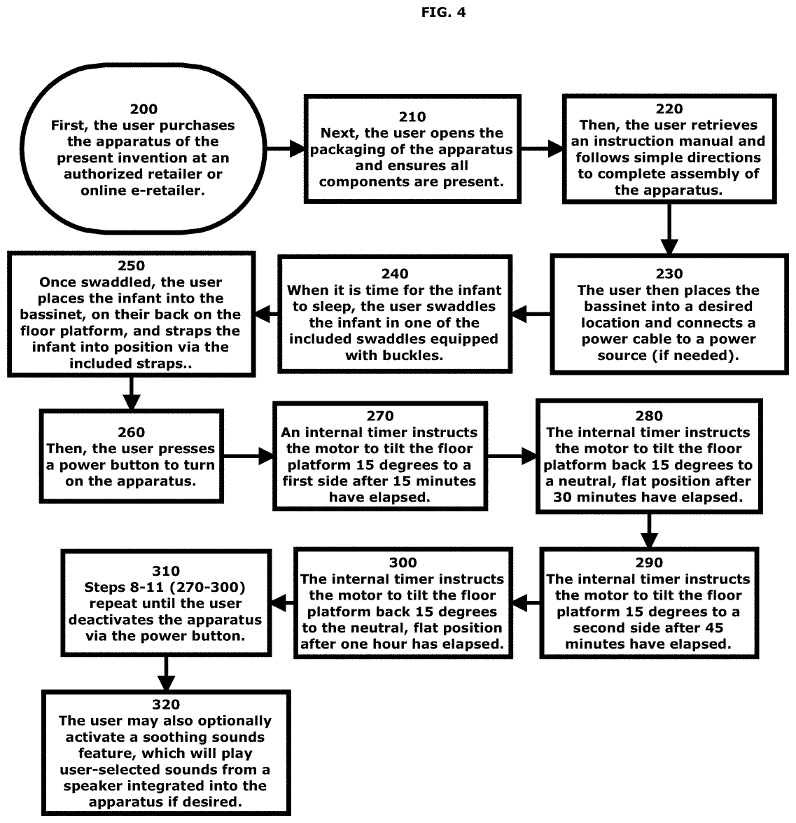

The process of use of the apparatus of the present invention, as shown in FIG. 4, is preferably as follows:
1. First, the user purchases the apparatus of the present invention at an authorized retailer or online e-retailer. (200)
2. Next, the user opens the packaging of the apparatus and ensures all components are present. (210)
3. Then, the user retrieves an instruction manual and follows simple directions to complete assembly of the apparatus. (220)
4. The user then places the bassinet into a desired location and connects a power cable to a power source (if needed). (230)
5. When it is time for the infant to sleep, the user swaddles the infant in one of the included swaddles equipped with buckles. (240)
6. Once swaddled, the user places the infant into the bassinet on their back on the floor platform and straps the infant into position via the included straps. (250)
7. Then, the user presses a power button to turn on the apparatus. (260)
8. An internal timer instructs the motor to tilt the floor platform 15 degrees to the first side after 15 minutes have elapsed. (270)
9. The internal timer instructs the motor to tilt the floor platform back 15 degrees to a neutral, flat position after 30 minutes have elapsed. (280)
10. The internal timer instructs the motor to tilt the floor platform 15 degrees to a second side after 45 minutes have elapsed. (290)
11. The internal timer instructs the motor to tilt the floor platform back 15 degrees to the neutral, flat position after one hour has elapsed. (300)
12. Steps 8-11 (270-300) repeat until the user deactivates the apparatus via the power button. (310)
13. The user may also optionally activate a soothing sounds feature, which will play user-selected sounds from a speaker integrated into the apparatus if desired. (320)

Alternate embodiments of the present invention may include variations in the size, shape, color, texture, pattern, and other conventional characteristics of the bassinet of the present invention. As such, it should be understood that the present invention may be made available in multiple designs and orientations; however, every embodiment is equipped with a pivoting bed, referenced as the floor platform (20), to facilitate the treatment and prevention of plagiocephaly in infants.

Having illustrated the present invention, it should be understood that various adjustments and versions might be implemented without venturing away from the essence of the present invention. Further, it should be understood that the present invention is not solely limited to the invention described in the embodiments above but further comprises any and all embodiments within the scope of this application.

The foregoing descriptions of specific embodiments of the present invention have been presented for purposes of illustration and description. They are not intended to be exhaustive or to limit the present invention to the precise forms disclosed. Many modifications and variations are possible in light of the above teaching. The exemplary embodiment was chosen and described to best explain the principles of the present invention and its practical application, thereby enabling others skilled in the art to best utilize the present invention and various embodiments with various modifications suited to the particular use contemplated.

I claim:

1. A bassinet that pivots, comprising:
a floor platform disposed in communication with a base;
a reciprocating rod descending from said base;
wherein said reciprocating rod is attached to a strap at an anchor point on said base;
an oscillatory drive disposed inside said base;
a swivel hinge on said floor platform;
retaining straps disposed in communication with said floor platform;
a motor disposed in communication with said floor platform; and
an actuating arm in communication with said motor.

2. The bassinet of claim 1 further comprising:
a power source, said motor disposed in communication with said power source;
a support for said motor;
wherein said actuating arm is disposed in communication with said floor platform,
wherein said actuating arm is configured to tilt said floor platform per revolutions of said motor; and
a controlling timer disposed in communication with said motor and said power source.

3. The bassinet of claim 1, wherein said motor is attached at a bottom of said platform.

4. The bassinet of claim 1, further comprising:
a battery disposed in communication with said motor;
a vibrating feature disposed in communication with said motor; and
a control circuit disposed in communication with said motor.

5. The bassinet of claim 1, wherein said floor platform is disposed in communication with said motor; and
wherein said motor disposed in communication with an offset link.

6. The bassinet of claim 1, wherein said actuating arm is in communication to a control of the stroke length and reciprocating motion.

7. The bassinet of claim 1, further comprising:
a crank device driven by a worm gear, and;
an upper end of said reciprocating arm coupled to said crank device.

8. The bassinet of claim 7, wherein said crank device is driven by a worm gear.

9. The bassinet of claim 8, further comprising:
an upper end of the reciprocating arm coupled to said crank device.

10. The bassinet of claim 1, further comprising:
a side wall configured to receive a swaddle in communication with said platform.

11. The bassinet of claim 1, further comprising:
a retaining strap configured to receive a onesie in communication with said platform.

12. The bassinet of claim 2, wherein said motor is attached at a bottom of said platform.

13. The bassinet of claim 2, further comprising:
a battery disposed in communication with said motor;
a vibrating feature disposed in communication with said motor; and
a control circuit disposed in communication with said motor.

14. The bassinet of claim 2, wherein said floor platform is disposed in communication with said motor; and
wherein said motor disposed in communication with an offset link.

15. The bassinet of claim 2, wherein said actuating arm is in communication to a control of the stroke length and reciprocating motion.

16. The bassinet of claim 2, further comprising:
a crank device driven by a worm gear, and;
an upper end of said reciprocating arm coupled to said crank device.

17. The bassinet of claim 16, further comprising:
an upper end of the reciprocating arm coupled to said crank device; and
wherein said crank device is driven by a worm gear.
